(19) United States
(12) Patent Application Publication
(10) Pub. No.: US 2019/0216549 A1
(43) Pub. Date: Jul. 18, 2019

(54) SURGICAL LIGHT HAVING AN OPERATING DEVICE

(71) Applicant: Karl Leibinger Medizintechnik GmbH & Co. KG, Mühlheim (DE)

(72) Inventor: Joachim Strölin, Mühlheim (DE)

(21) Appl. No.: 16/332,302

(22) PCT Filed: Aug. 11, 2017
(86) PCT No.: PCT/EP2017/070488

§ 371 Date: Mar. 11, 2019

(30) Foreign Application Priority Data
Sep. 12, 2016 (DE) 10 2016 117 068.2

(51) Int. Cl. A61B 34/00; A61B 90/35; A61B 90/00; F21V 21/29; F21V 21/15

(52) U.S. Cl. CPC A61B 34/25; A61B 90/35; A61B 2034/2048; F21V 21/29; F21V 21/15; A61B 90/37

(57) ABSTRACT

The invention relates to a surgical light comprising a lamp unit having a plurality of individual light sources accommodated in a housing, a retaining arm system which receives the lamp unit in a displaceable and/or pivotable manner, and an operating device for inputting a change command regarding a light adjustment of the lamp unit. According to the invention, the operating device has a touch-sensitive screen and is pivotally mounted at a fastening section provided on the retaining arm system.

The invention relates to a surgical light comprising a lamp unit having a plurality of individual light sources accommodated in a housing, a retaining arm system which receives the lamp unit in a displaceable and/or pivotable manner, and an operating device for inputting a change command regarding a light adjustment of the lamp unit.

From the state of the art, generic surgical lights are basically known already. For example, U.S. Pat. No. 6,880,957 B2 discloses a lighting device with electronic shadow compensation.

However, in the configurations known from the state of the art it has turned out to be a drawback that, especially when the operating device is attached to the housing of the lamp unit, the position of the lamp unit is frequently inadvertently changed when the operating device is actuated. This is especially due to the fact that the retaining arm systems used are relatively smooth-running. When the lamp unit is gripped, it is intended to be swiveled as easily as possible. Therefore, it has been considered already to mount the operating devices basically at positions other than the lamp unit, such as at the retaining arm system. It has turned out, however, that for actuating the operating devices used, excessively high operating forces still have to be applied which may result in inadvertent displacement of the surgical light/lamp unit during operation. Inadvertent displacement of the surgical light occurs especially when the height of the operating device is not optimally adjusted to the operator.

Therefore, it is the object of the present invention to eliminate said drawbacks known from the state of the art and, especially, to provide a surgical light in which during operation a position of the lamp unit is intended to be minimally influenced by the operating input of the user.

According to the invention, this is achieved by the fact that the operating device includes a touch-sensitive screen/touchscreen and is pivotably mounted at a fastening section provided at the retaining arm system.

Such arrangement and design of the operating device, on the one hand, helps to optimally adjust the position of the operating device to the respective operator and, on the other hand, to actuate the same with minimum effort. This helps to significantly improve the operation of the surgical light.

Further advantageous embodiments are claimed in the subclaims and shall be illustrated hereinafter.

When the operating device is arranged in the area of a universal joint of the retaining arm system, preferably at a supporting arm portion of the universal joint forming a gimbal bracket, the probability of inadvertently changing the position of the lamp unit when the operating device is actuated is further reduced.

When the fastening section forms a distal end of a supporting arm portion of the retaining arm system, the operating device is preferably freely accessible for actuation by the operator.

Moreover, it is advantageous when a user interface of the touch-sensitive screen is (spatially) identically oriented both in a first position and in a second position of the operating device. In this way, the operation is further facilitated.

In this context, it is especially useful when the operating device in the first position is arranged with its center of gravity, preferably a center of volume or mass, above the fastening section, when viewed in a vertical direction, and/or in the second position is arranged with the center of gravity below the fastening section, when viewed in the vertical direction. This helps to permit especially ergonomic operation.

In terms of control it is especially advantageous when the touch-sensitive screen is coupled to a control unit (for data transmission), wherein the control unit preferably is coupled in turn to a position detection sensor/acceleration sensor, and wherein the control unit acts to control the touch-sensitive screen so that the user interface of the touch-sensitive screen in the operating state maintains a fixed (spatial) orientation while the operating device is swiveled. In this way, the operation of the operating device and of the surgical light is further facilitated.

Moreover, it is of advantage when the operating device is pivotably mounted between at least two positions offset against (about) 180° which preferably form the first and second positions. This helps to further facilitate operation and the operating device can be individually adjusted to the size of the operator.

In this context, it is also useful when the operating device is (detachably) secured/fixed in position positively and/or non-positively to the retaining arm system in at least two positions, preferably the first position and the second position. Thus, the two positions are configured as locking positions and the operating device is safely supported.

Further, it is advantageous when the operating device includes a box-shaped operating housing which is formed to be separated in terms of material from the operating area. In this context, it is especially advantageous when a longitudinal axis of the operating device is vertically oriented in each of the at least two positions of the operating device. This helps to further facilitate the structure of the operating device.

It is of particular advantage when the operating device/the operating housing is configured as to thickness so that it can be gripped by a human hand. This helps to further facilitate operation.

In this context, it is especially advantageous when the operating device is mounted to the fastening section so that in any position/swivel position relative to the retaining arm system it can be gripped from behind (by a human hand) in a section adjacent to the fastening section. This means that in any position at least a clearance of at least 2 cm, preferably at least 3 cm, is always formed on a side of the operating device/the operating housing facing away from the touch-sensitive screen. In this way, the operating device has an especially ergonomic design and, in the form of a handle, can be gripped by the operating surgeon.

In other words, the surgical light according to the invention includes an operating device arranged on a surgical light which combines a touch-display surface (user interface) and a handle (operating housing/handle). The operating device may be in the form of a handle in such way that the operating functions can be operated by the operator's thumb and the operator's fingers lying there behind prevent the operating device from backing away. The operating device may be arranged to be rotatable about 180° (upwards or downwards) in its orientation so that it can be adapted to the size of the operator. The display (user interface) may be of the flip type. Thus, easy handling is possible with simultaneous positioning of the lamp.

Hereinafter, the invention shall be illustrated in detail by way of various figures; in this context, also various example configurations shall be described;

wherein:

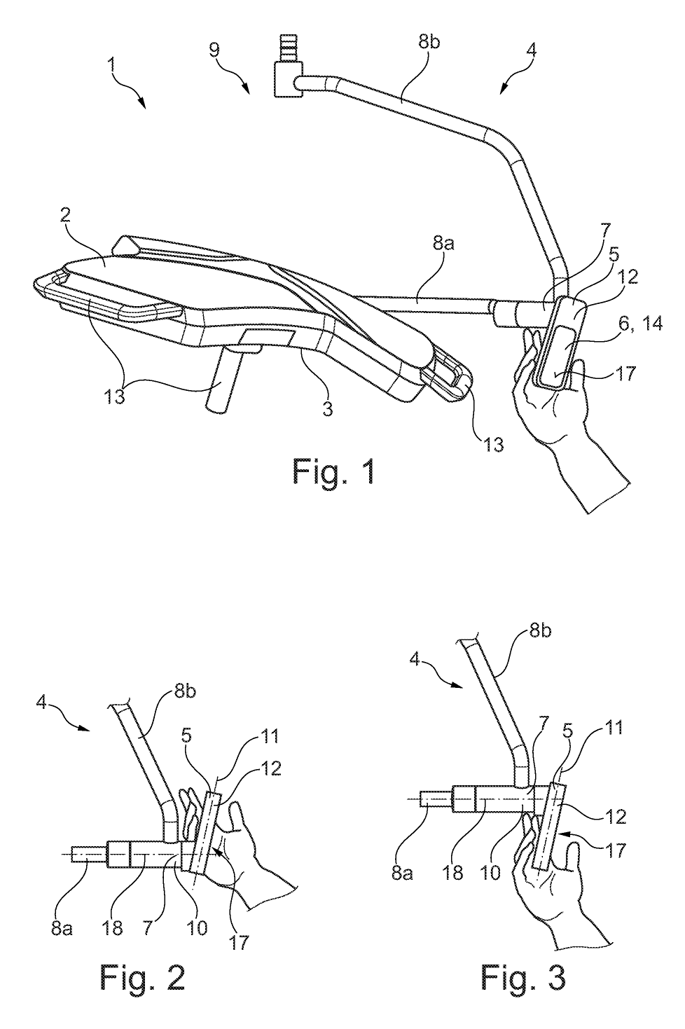

FIG. 1 shows a perspective lateral representation of the surgical light according to the invention as set forth in a preferred example configuration, wherein especially an operating device and mounting thereof at a retaining arm system of the surgical light are evident,

FIG. 2 shows a detailed lateral representation of the surgical light according to FIG. 1 in the area of the operating device which is arranged in a first position relative to the retaining arm system,

FIG. 3 shows a detailed lateral representation of the surgical light according to FIG. 1, similarly to FIG. 2, with the operating device now being swiveled in a second position rotated against the first position by about 180°,

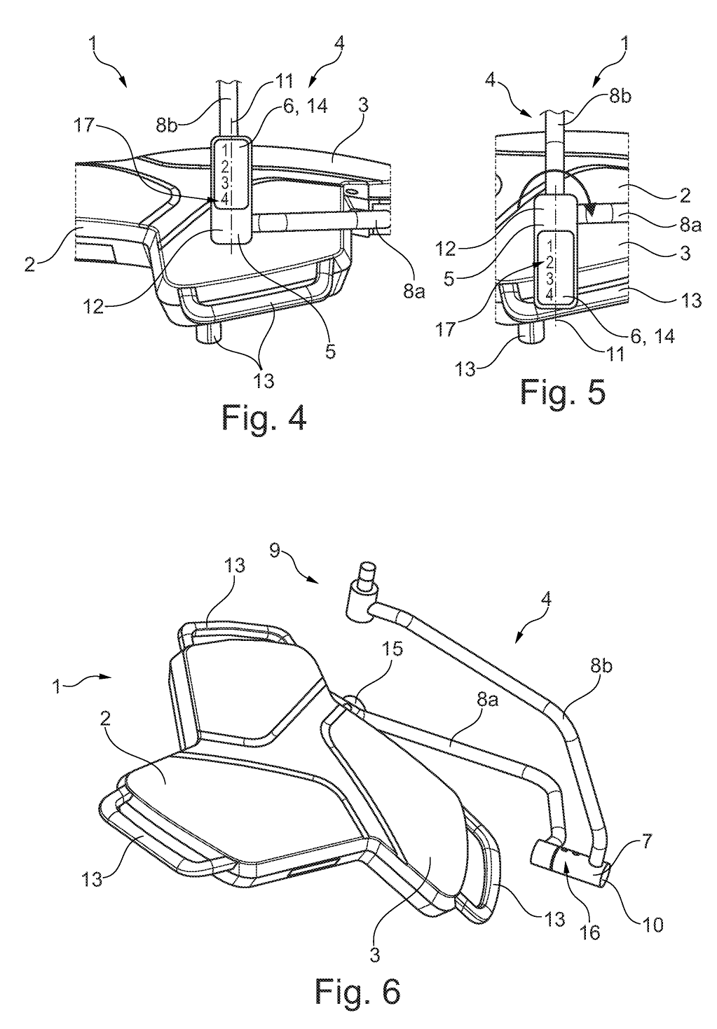

FIG. 4 shows a detailed front view of the operating device mounted at the retaining arm system in the first position according to FIG. 2,

FIG. 5 shows a detailed front view of the operating device mounted at the retaining arm system in the second position according to FIG. 3, and

FIG. 6 shows a perspective representation of the surgical light when the operating device is removed so that the design of a fastening section of the retaining arm system accommodating the operating device is clearly visible.

The Figures are merely schematic and serve exclusively for the comprehension of the invention. Like elements are provided with like reference numerals.

A surgical light according to the invention is illustrated in FIG. 1 according to a preferred example configuration. The surgical light 1 is designed for mounting to a ceiling of an operating room of a hospital. For this purpose, the surgical light 1 includes a retaining arm system 4. Said retaining arm system 4 receives a lamp unit 2 in a pivotable and displaceable manner. The retaining arm system 4 is further connected to a (first) end section facing away from the lamp unit 2 in operation with an anchoring element of the surgical light 1 which is not shown here in detail for the sake of clarity. The anchoring element then typically serves for mounting the surgical light 1 to the ceiling of the operating room.

In FIG. 6, the surgical light 1 according to the invention is initially illustrated without an operating device, as it is mounted in FIG. 1 and described in detail in the following. From FIG. 6 equally the basic extension of part of the retaining arm system 4 is evident. For the sake of clarity, the retaining arm system 4 is not shown completely but merely in the area of two supporting arm portions 8a and 8b. Both supporting arm portions 8a and 8b form a universal joint 9/a gimbal with two swivel joints 15 and 16. A first supporting arm portion 8a is pivotably/rotatably attached to the lamp unit 2 via a first swivel joint 15. In particular, said first supporting arm portion 8a is rotatably arranged on a housing 3 of the lamp unit 2. Thus, the housing 3 in turn is pivotably received at the first supporting arm portion 8a, with the first supporting arm portion 8a forming a second end section of the retaining arm system 4 facing away from the first end section on the side of the first swivel joint 15.

From the first swivel joint 5 the first supporting arm portion 8a extends to a (second) swivel joint 16 facing away from the housing 3. By means of the second swivel joint 16 a second supporting arm portion 8b is pivotally connected to the first supporting arm portion 8a. The two swivel joints 15 and 16 form pivot axes which are aligned substantially normal to each other. The second supporting arm portion 8b thus forms the universal joint 9 by interaction with the first supporting arm portion 8a as well as the two swivel joints 15 and 16. Consequently, the restraining arm system 4 is also referred to as gimbal retaining arm system 4.

The second supporting arm portion 8b is further connected during operation by its side again facing away from the second swivel joint 16 to a third supporting arm portion of the retaining arm system 4 not illustrated here for the sake of clarity, for example while a further third swivel joint/and/or a further (second) universal joint is/are formed.

The housing 3 of the lamp unit 2 has a substantially umbrella-type configuration, as is evident from FIGS. 1 and 6. The housing 3 extends substantially curved/bent along an imaginary ball segment surface. The housing 3 thus is umbrella-shaped. In FIG. 6 the lamp unit 2 is depicted from an upper side. The upper side takes a convex shape. At the lower side of the housing 3 facing away from the upper side, plural light sources not detailed here for the sake of clarity are arranged in the housing 3 in the usual manner. Each of the light sources is formed by an LED module preferably including at least one LED as well as lens optics associated with said at least one LED. The different light sources are arranged to be spread in/at the housing 3 and, in the energized state, produce a respective directed light beam which is emitted from the housing toward the lower side of the housing 3.

It is also especially clearly visible in FIG. 1 that an operating device 5 is mounted at the second supporting arm portion 8b forming a gimbal. Said operating device 5 is pivotably/rotatably mounted at said second supporting arm portion 8b. In this context, it is outlined that in further example configurations said operating device 5 is also mounted at other supporting arm portions of the retaining arm system 4, such as at the first supporting arm portion 8a.

The operating device 5 is substantially box-shaped. In a box-shaped operating housing 12 of the operating device 5, a touch-sensitive screen 6 (also referred to as touchscreen/touch display) is mounted. As is evident from FIGS. 4 and 5, the operating housing 12/operating device 5 includes a rectangular front 17. The operating device 5, i.e. the operating housing 12 thereof, is pivotably/rotatably mounted at the retaining arm system 4, viz. at the second supporting arm portion 8b. For this purpose, the second supporting arm portion 8b includes a (tubular) fastening section 7 which is formed at a distal end 10 of the second supporting arm portion 8b. The distal end 10 is clearly visible also in FIG. 6.

In this example configuration, the operating device 5 is secured in position relative to the second supporting arm portion 8b in two positions/swivel positions according to FIGS. 2 and 3 and, resp., 4 and 5. A first position, as is evident from FIG. 2, for example, is the position in which the operating device 5 is vertically oriented and a center of volume of the operating device 5 is disposed above the fastening section 7. The operating device 5 is arranged, especially above its swivel axis 18 formed by a longitudinal axis of the fastening section 7, in a first position. In said first position the operating device 5/operating housing 12 is positively supported/fixed at the fastening section 7 by a specific retaining force. Alternatively, however, also a non-positive and, resp., a positive and non-positive support is provided.

In FIGS. 3 and 5, a second position of the operating device 5 is realized. The operating housing 12 is positively (alternatively also non-positively or positively and non-positively) supported on the fastening section 7 even in said second position by a specific retaining force. The retaining mechanism required for this purpose is not shown in detail for the sake of clarity, but it is integrated in the connecting area between the operating housing 12 and the fastening section 7. Thus, the retaining mechanism is provided at an interface between the fastening section 7 and the operating housing 12. The second position is rotated about 180° vis-à-vis the second position. Therefore, the center of volume of the operating device 5 in said second position is below the fastening section 7/the swivel axis 18.

In the first position according to FIG. 4, the touch-sensitive screen 6 is substantially vertically oriented. The operating device 5 extending longitudinally, approximately along a longitudinal axis 11, therefore is vertically oriented in the first position. A user interface 14/touch surface of the touch-sensitive screen 6 is also oriented vertically (with respect to a fixed imaginary coordinate system) in said first position.

As is clearly evident in connection with FIG. 5, the user interface 14 can be rotated relative to the operating device/operating housing 12 when the operating device 5 is rotated.

Especially, the user interface 14 is oriented identically in space both in the first position and in the second position. In the second position, too, the operating device 5 is vertically oriented.

The operating device 5/operating housing 12 is dimensioned and arranged to be spaced apart from the remaining second supporting arm portion 8b adjacent to the fastening section 7 such that a human hand can grip behind the same, as is clearly evident from FIGS. 2 and 3, in the first position as well as in the second position. In this example configuration, it is possible to grip behind the operating device even in any position of 360° around the axis of rotation 18. In addition, the thickness of the operating device 5/operating housing 12 is selected so that the latter can be gripped by a human hand. Therefore, the operating device 5 as a whole is configured as a handle 13.

The touch-sensitive screen is coupled to a control unit not detailed here for the sake of clarity, wherein the control unit in turn is operatively connected to a position detection sensor, such as an acceleration sensor, which is not detailed here for the sake of clarity, either. Said acceleration sensor is equally provided in the operating device 5. The position detection sensor is configured so that it detects a position of the operating device 5 in space and transmits a corresponding measured value to the control unit. In response to the measured value, the control unit then acts upon the touch-sensitive screen so that the user interface 14 is retained in its fixed spatial orientation in the operating state, when the operating device 5 is swiveled, but in its orientation relative to the operating housing 12 is equally swiveled about 180°.

### LIST OF REFERENCE NUMERALS

1 surgical light
2 lamp unit
3 housing
4 retaining arm system
5 operating device
6 touch-sensitive screen
7 fastening section
8a first supporting arm portion
8b second supporting arm portion
9 universal joint
10 distal end
11 longitudinal axis
12 operating housing
13 handle
14 user interface
15 first swivel joint
16 second swivel joint
17 front
18 axis of rotation

1-10. (canceled)

11. A surgical light comprising a lamp unit having a plurality of individual light sources accommodated in a housing, a retaining arm system which receives the lamp unit in a displaceable and/or pivotable manner, and an operating device for inputting a change command regarding a light adjustment of the lamp unit, wherein the operating device has a touch-sensitive screen and is pivotally mounted at a fastening section provided on the retaining arm system.

12. The surgical light according to claim 11, wherein the operating device is mounted in the area of a universal joint of the retaining arm system.

13. The surgical light according to claim 12, wherein the fastening section forms a distal end of a supporting arm portion of the retaining arm system.

14. The surgical light according to claim 11, wherein the fastening section forms a distal end of a supporting arm portion of the retaining arm system.

15. The surgical light according to claim 14, wherein a user interface of the touch-sensitive screen is oriented identically in a first position and in a second position of the operating device.

16. The surgical light according to claim 12, wherein a user interface of the touch-sensitive screen is oriented identically in a first position and in a second position of the operating device.

17. The surgical light according to claim 11, wherein a user interface of the touch-sensitive screen is oriented identically in a first position and in a second position of the operating device.

18. The surgical light according to claim 11, wherein in the first position the operating device is arranged with its center of gravity above the fastening section when viewed in a vertical direction and/or in the second position is arranged with its center of gravity below the fastening section when viewed in a vertical direction.

19. The surgical light according to claim 17, wherein the touch-sensitive screen is coupled to a control unit, wherein the control unit acts to control the touch-sensitive screen in such manner that the user interface of the touch-sensitive screen maintains a fixed orientation in the operating state when the operating device is swiveled.

20. The surgical light according to claim 19, wherein in the first position the operating device is arranged with its center of gravity above the fastening section when viewed in a vertical direction and/or in the second position is arranged with its center of gravity below the fastening section when viewed in a vertical direction.

21. The surgical light according to claim 11, wherein the operating device is pivotably mounted between at least two positions offset against each other by approx. 180°.

22. The surgical light according to claim 11, wherein in at least two positions the operating device is secured in position positively and/or non-positively on the retaining arm system.

23. The surgical light according to claim 22, wherein the operating device includes a box-shaped operating housing which is formed to be separated in terms of material from the fastening section.

24. The surgical light according to claim 11, wherein the operating device includes a box-shaped operating housing which is formed to be separated in terms of material from the fastening section.

25. The surgical light according to claim 23, wherein the operating device is mounted at the fastening section so that in any position relative to the retaining arm system the operating device can be gripped from behind.

26. The surgical light according to claim 11, wherein the operating device is mounted at the fastening section so that in any position relative to the retaining arm system the operating device can be gripped from behind.

27. An apparatus comprising:
a lamp unit having a plurality of individual light sources accommodated in a housing;
a retaining arm system which receives the lamp unit in a displaceable and/or pivotable manner; and
an operating device configured to receive a change command regarding a light adjustment of the lamp unit, wherein the operating device has a touch-sensitive screen and is pivotally mounted at a fastening section provided on the retaining arm system.

28. The apparatus of claim 27, wherein the operating device is mounted in the area of a universal joint of the retaining arm system.

29. The apparatus of claim 27, wherein in the first position the operating device is arranged with its center of gravity above the fastening section when viewed in a vertical direction and/or in the second position is arranged with its center of gravity below the fastening section when viewed in a vertical direction.

30. An apparatus comprising:
a lamp unit having a plurality of individual light sources accommodated in a housing;
a retaining arm system which receives the lamp unit in a displaceable and/or pivotable manner; and
an operating device configured to receive a change command regarding a light adjustment of the lamp unit,
wherein the operating device has a touch-sensitive screen and is pivotally mounted at a fastening section provided on the retaining arm system,
wherein the operating device is mounted in the area of a universal joint of the retaining arm system,
wherein the fastening section forms a distal end of a supporting arm portion of the retaining arm system,
wherein a user interface of the touch-sensitive screen is oriented identically in a first position and in a second position of the operating device,
wherein in the first position the operating device is arranged with its center of gravity above the fastening section when viewed in a vertical direction and/or in the second position is arranged with its center of gravity below the fastening section when viewed in a vertical direction,
wherein the touch-sensitive screen is coupled to a control unit, wherein the control unit acts to control the touch-sensitive screen in such manner that the user interface of the touch-sensitive screen maintains a fixed orientation in the operating state when the operating device is swiveled,
wherein the operating device is pivotably mounted between at least two positions offset against each other by approximately 180°,
wherein in at least two positions the operating device is secured in position positively and/or non-positively on the retaining arm system,
wherein the operating device includes a box-shaped operating housing which is formed to be separated in terms of material from the fastening section,
wherein the operating device is mounted at the fastening section so that in any position relative to the retaining arm system the operating device can be gripped from behind.
